(19) United States
(12) Patent Application Publication
(10) Pub. No.: US 2003/0150009 A1
(43) Pub. Date: Aug. 7, 2003

(54) Generation of multiple embryo maize

(75) Inventors: Daniel R. Gallie, Riverside, CA (US); Todd E. Young, Riverside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The University of California

(21) Appl. No.: 10/072,077

(22) Filed: Feb. 7, 2002

(51) Int. Cl. A01H 5/00; A01H 1/00; C12N 15/82

(52) U.S. Cl. 800/278; 800/320.1

(57) ABSTRACT

The present invention is generally related to plant genetic engineering. In particular, the invention provides methods of inhibiting programmed cell death in maize and other grasses. This invention also provides transgenic maize plants having kernels with multiple embryos and kernels from transgenic maize plants having multiple embryos.

### FIELD OF THE INVENTION

The present invention is generally related to plant genetic engineering. In particular, the invention provides methods of inhibiting programmed cell death in the lower floret of a maize plant and in other grasses. This invention also provides transgenic maize plants having kernels with multiple embryos and kernels from transgenic maize plants having multiple embryos.

### BACKGROUND OF THE INVENTION

In 1993, there were more than 72.7 million acres planted with corn for grain production in the U.S. Maize grown in the U.S. is predominantly of the yellow dent type, a commodity crop largely used to feed domestic animals, either as grain or silage. The remainder of the crop is exported or processed by wet or dry milling to yield products such as high fructose maize syrup and starch or oil, grits and flour. These processed products are used extensively in the food industry, for example, maize starch serves as a raw material for an array of processed foods, and in industrial manufacturing processes. Other corn products include corn oil, corn syrup, dextrose, maltodextrins, and ethanol. The by-products from these processes are often used in animal feeds and in a vast array of food additives and consumer products. In 2100, 24 percent of all harvested crop acres were harvested as corn for grain for a total crop value of $18.44 billion, up from $17.93 billion in 1999. This is greater in dollar value than any other crop grown in the U.S. For instance, the value of the soybean harvest in year 200 was 13.6 billion whereas wheat was 5.89 billion. Corn is grown in more countries than any other crop and is a major source of food and protein for both humans and animals throughout the world. Because of the value of corn to the U.S., there is a continuous and substantial effort to increase its starch, protein and oil content and because of the multitude of products extracted from corn, corn varieties that are either high oil, high starch, or high protein have been developed. Most of the oil and protein in maize kernels is present in the embryo.

Maize produces unisexual flowers, or florets, that are physically separated on the plant. The male flowers that produce pollen develop on the male inflorescence, at the top of the plant, whereas the female flowers from which the kernels develop, are present on the female inflorescence. The florets on the ear produce female but not male floral organs. Following the early differentiation of the ear, pairs of spikelets develop along the length of the ear. Within each spikelet, two floret primordia develop. Each floret primordium further develops into initials for a lemma, a plaea, two lodicules, three stamens, and a central gynoecium. Of the two florets produced in each spikelet in the maize ear, the lower one dies and the upper one develops into a kernel. Of the remaining floret, the three stamens also abort, leaving only the central gynoecium to develop into a mature ovary, that, following pollination, results in the development of the kernel.

The death of the lower floret in each spikelet is one example of programmed cell death during the development of the maize plant. Other examples include the death of the endosperm, the tissue of the kernel in which the bulk of starch synthesis and deposition occurs, during the late development of the kernel. Programmed cell death is initiated and controlled by the balance of several plant growth regulators. For instance, the cell death of the maize endosperm is promoted by the hormone ethylene but is delayed by the hormone abscisic acid. Young et al., Plant Mol. Biol. 42: 397-414 (2000), Young et al., Plant Physiol. 115:737-751 (1997). Hormonal control of cell death also applies to floral organ cell death. The abortion of the male floral organs within the florets of the ear (the stamens) involves a programmed cell death that requires the hormone gibberellic acid. Calderon-Urrea et al., Development 126, 435-441 (1999).

Inhibiting senescence in a plant has been identified as a way to prolong the photosynthetically active life-span of a plant. Cytokinin is an enzyme known to inhibit leaf senescence. Plants with altered senescence patterns have leaves that retain high levels of chlorophyll throughout seed and flower development. Tobacco plants with altered leaf senescence patterns have enhanced yield of biomass and flower and seed production even though seed yield per flower remains the same. (See U.S. Pat. No. 5,689,042).

A need exists for new methods of increasing food production. The present invention addresses these and other needs by providing methods of inhibiting programmed cell death in maize and other grasses.

### SUMMARY OF THE INVENTION

The present invention relates to methods of inhibiting programmed cell death in the lower floret of a maize plant and in other grasses.

In one embodiment, the invention provides a method of inhibiting programmed cell death in a maize plant. The method include introducing a construct comprising a programmed cell death inducible promoter operably linked to a nucleotide sequence that inhibits programmed cell death into the maize plant. In particular, the programmed cell death in the lower floret of the maize plant is inhibited.

In some embodiments of the invention, the nucleotide sequence encodes a plant growth regulator synthesizing enzyme. In one embodiment, the plant growth regulator synthesizing enzyme catalyzes the synthesis of cytokinin. In yet another embodiment of the invention, the plant growth regulator synthesizing enzyme is isopentenyl transferase.

In some embodiments of the invention, the programmed cell death inducible promoter is SAG12. In one embodiment, the SAG12 promoter is from Arabidopsis thaliana. In another embodiment, the SAG12 promoter is 70% identical to SEQ ID NO:1.

In some embodiments of the invention, the method of inhibiting programmed cell death in a maize plant includes detecting increased levels of protein within the plant.

In some embodiments, the method of inhibiting programmed cell death in a maize plant includes detecting increased levels of oil within the plant. In other embodiments, the method of inhibiting programmed cell death in a maize plant includes detecting increased levels of oil and protein within said plant. In yet other embodiments, the method of inhibiting programmed cell death in a maize plant includes detecting the presence of a kernel having multiple embryos.

In one aspect of the invention, the construct is introduced by a type of sexual cross. In another aspect, the construct is introduced by transformation.

In some embodiment, this invention provides a transgenic maize plant comprising an expression cassette comprising a programmed cell death-inducible promoter operably linked to a nucleotide sequence encoding an inhibitor of programmed cell death, the maize plant having kernels with multiple embryos.

In some embodiments, the nucleotide sequence encodes a plant growth regulator synthesizing enzyme. In one embodiment, the enzyme catalyzes the synthesis of cytokinin. In another embodiment the enzyme is isopentenyl transferase.

In some embodiments, the programmed cell death inducible promoter is SAG12.

In some embodiments, this invention provides a kernel from a transgenic maize plant comprising multiple embryos, wherein the kernel has increased oil and protein content.

In some embodiments, the method of inhibiting programmed cell death in a maize plant includes introducing a promoter from a floret specific gene operably linked to a nucleotide sequence that inhibits programmed cell death into said plant, whereby programmed cell death in the lower floret of said plant is inhibited. In one aspect, the floret specific gene is associated with programmed cell death. In another aspect, the floret specific gene is not associated with programmed cell death.

In some embodiments, the nucleotide sequence encodes a plant growth regulator synthesizing enzyme. In one aspect, the enzyme catalyzes the synthesis of cytokinin. In another aspect, the enzyme is isopentenyl transferase. In yet another aspect, the method of inhibiting programmed cell death includes detecting increased levels of oil and protein within the maize plant. In even yet another aspect, the method of inhibiting programmed cell death includes detecting the presence of a kernel having multiple embryos.

### DETAILED DESCRIPTION OF THE INVENTION

I. Introduction

This invention demonstrates for the first time a method of inhibiting programmed cell death in the lower floret of a maize plant and in other grasses. Grasses may include but are not limited to grasses such as wheat, rye, rice, sorghum, or oat. This invention also provides for the first time a maize plant having kernels with multiple embryos. Because most of the oil and protein in maize kernels is present in the embryo, maize plants with multiple embryonic kernels contain more protein and oil than maize plants having kernels with one embryo. A maize plant having kernels with multiple embryos is therefore, valuable as a food source and commodity.

In an embodiment of this invention, a construct comprising a programmed cell death inducible promoter linked to a nucleotide sequence that inhibits programmed cell death, is introduced into maize or other grasses. In transgenic maize plants containing this construct, the programmed cell death inducible promoter is activated during the programmed cell death of the lower floret in the spikelets of the maize plant. The programmed cell death of the lower floret is inhibited by expression of the protein encoded by the nucleotide sequence and the surviving lower floret produces an embryo after pollination. The kernel developing in the upper floret and the embryo from the lower floret fuse together, thereby producing a kernel composed of two or more embryos attached to a normal sized endosperm. Germination of the double embryo kernel results in the growth of two or more distinct, healthy, and fertile maize plants. This invention, therefore, demonstrates for the first time a maize plant with multiple embryonic kernels that increase the oil and protein content of maize plants.

II. Definitions

The term “programmed cell death” refers to a mode of cell death. There are two general modes of cell death, programmed cell death and necrosis. In necrosis, the cell is a passive victim of various forms of trauma causing loss of membrane integrity. In contrast, programmed cell death requires de novo gene expression and is characterized by changes in nuclear morphology, activation of nucleases and proteases, and internucleosomal degradation of nuclear DNA. In programmed cell death, the cell is shutting down according to a controlled pattern of events during which cells undergo distinct metabolic and structural changes prior to cell death. Programmed cell death is an essential process for normal development and homeostasis in multicellular organisms such as mammals, insects and plants. Programmed cell death can occur with or without a proccess of aging. For example, the lower floret of the maize plant undergoes programmed cell death while still quite young and never undergoes an aging process.

Typically, the term “senescence” refers to the process of aging that may occur before cell death.

The term “programmed cell death associated gene” refers to a gene involved in a programmed cell death pathway. A programmed cell death associated gene is a gene whose expression may be induced at various times in the programmed cell death pathway. Programmed cell death associated genes may be induced during premature onset of programmed cell death or during regular onset of programmed cell death. They may be induced in the beginning stages, middle stages or end stages of programmed cell death. Programmed cell death associated genes may also include a gene whose expression is induced during senescence, e.g., senescence-associated genes. Senescence associated genes are expressed during senescence.

The term “programmed cell death inducible promoter” refers to a promoter from a gene whose expression is induced during programmed cell death. The term programmed cell death inducible promoter may refer to a promoter from a gene whose expression is induced in the beginning stages of programmed cell death. A programmed cell death inducible promoter is capable of preferentially promoting gene expression in a plant tissue in a developmentally regulated manner such that expression of a 3′ protein coding region occurs substantially only when the plant tissue is undergoing programmed cell death. The term programmed cell death-inducible promoter may include senescence inducible promoters (e.g., promoters from a gene induced in response to senescence, not including the see promoter, e.g., a SAG12 promoter).

The phrase “nucleic acid” refers to a single or double-stranded polymer of deoxyribonucleotide or ribonucleotide bases read from the 5′ to the 3′ end. Nucleic acids may also include modified nucleotides that permit correct read through by a polymerase and do not alter expression of a polypeptide encoded by that nucleic acid.

The phrase “polynucleotide sequence” or “nucleic acid sequence” includes both the sense and antisense strands of a nucleic acid as either individual single strands or in the duplex. It includes, but is not limited to, self-replicating plasmids, chromosomal sequences, and infectious polymers of DNA or RNA.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

As used herein, the term “promoter” includes all sequences capable of driving transcription of a coding sequence in a plant cell. Thus, promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is different from any naturally occurring allelic variants.

A polynucleotide “exogenous to” an individual plant is a polynucleotide which is introduced into the plant by any means other than by a sexual cross. Examples of means by which this can be accomplished are described below, and include Agrobacterium-mediated transformation, biolistic methods, electroporation, in planta techniques, and the like. Such a plant containing the exogenous nucleic acid is referred to here as an R₁ generation transgenic plant. Transgenic plants which arise from sexual cross or by selfing are descendants of such a plant.

As used herein, a homolog of a particular embryo-specific gene is a second gene in the same plant type or in a different plant type, which has a polynucleotide sequence of at least 50 contiguous nucleotides which are substantially identical (determined as described below) to a sequence in the first gene. It is believed that, in general, homologs share a common evolutionary past.

A “polynucleotide sequence from” a gene is a subsequence or full length polynucleotide sequence of a gene which, when present in a transgenic plant, has the desired effect, for example, inhibiting expression of the endogenous gene driving expression of an heterologous polynucleotide. A full length sequence of a particular gene disclosed here may contain about 95%, usually at least about 98% of an entire sequence shown in the Sequence Listing, below.

The term “reproductive tissues” as used herein includes fruit, ovules, seeds, pollen, pistols, flowers, or any embryonic tissue.

In the case of both expression of transgenes and inhibition of endogenous genes (e.g., by antisense, or sense suppression) one of skill will recognize that the inserted polynucleotide sequence need not be identical and may be “substantially identical” to a sequence of the gene from which it was derived. As explained below, these variants are specifically covered by this term.

In the case where the inserted polynucleotide sequence is transcribed and translated to produce a functional polypeptide, one of skill will recognize that because of codon degeneracy a number of polynucleotide sequences will encode the same polypeptide.

In the case of polynucleotides used to inhibit expression of an endogenous gene, the introduced sequence need not be perfectly identical to a sequence of the target endogenous gene. The introduced polynucleotide sequence will typically be at least substantially identical (as determined below) to the target endogenous sequence.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison may be conducted by the local homology algorithm of Smith and Waterman Add. APL. Math. 2:482 (1981), by the homology alignment algorithm of Needle man and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 70% sequence identity, at least 80% sequence identity, preferably at least 85%, more preferably at least 90% and most preferably at least 95%, compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%, preferably at least 60%, 70%, 80% or more preferably at least 90%, and most preferably at least 95%. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

Another indication that nucleotide sequences are substantially identical is if two molecules hybridize to each other, or a third nucleic acid, under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 molar at pH 7 and the temperature is at least about 60° C.

For the purposes of this disclosure, stringent conditions for hybridizations are those which include at least one wash in 0.2X SSC at 63° C. for 20 minutes, or equivalent conditions. Moderately stringent conditons include at least one wash (usually 2) in 0.2X SSC at a temperature of at least about 50° C., usually about 55° C., for 20 minutes, or equivalent conditions.

The term “expression cassette” refers to any recombinant expression system for the purpose of expressing a nucleic acid sequence of the invention in vitro or in vivo, constitutively or inducibly, in any cell, including, in addition to plant cells, prokaryotic, yeast, fungal, insect or mammalian cells. The term includes linear or circular expression systems. The term includes all vectors. The cassettes can remain episomal or integrate into the host cell genome. The expression cassettes can have the ability to self-replicate or not, i.e., drive only transient expression in a cell. The term includes recombinant expression cassettes which contain only the minimum elements needed for transcription of the recombinant nucleic acid.

As used herein, the term “operably linked,” refers to a functional relationship between two or more nucleic acid (e.g., DNA) segments. Typically, it refers to the functional relationship of a transcriptional regulatory sequence to a transcribed sequence. For example, a promoter (defined below) is operably linked to a coding sequence, such as a nucleic acid of the invention, if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant means” also encompass the ligation of nucleic acids having coding or promoter sequences from different sources into an expression cassette or vector for, e.g., expression of a fusion protein, or, inducible or constitutive expression of a protein (e.g., a promoter operably linked to a nucleic acid of the invention).

As used herein, the “sequence” of a gene (unless specifically stated otherwise) or nucleic acid refers to the order of nucleotides in the polynucleotide, including either or both strands (sense and antisense) of a double-stranded DNA molecule, e.g., the sequence of both the coding strand and its complement, or of a single-stranded nucleic acid molecule (sense or antisense). For example, in alternative embodiments, promoters drive the transcription of sense and/or antisense polynucleotide sequences of the invention.

III. Preparation of Programmed Cell Death Inducible Promoter

The programmed cell death inducible promoters of this invention are used to drive expression of genes that inhibit programmed cell death in maize or other grasses. One of skill in the art can readily identify programmed cell death inducible promoters by identifying genes whose expressions are induced during programmed cell death and determining the promoter region of those genes.

In order to identify genes involved in the programmed cell death pathway, standard techniques are used to identify cells undergoing programmed cell death. In one embodiment of the invention, the identified genes are expressed in the beginning stages of programmed cell death. In another embodiment, the identified genes are expressed during the premature onset of programmed cell death. One of skill can determine that a cell is undergoing programmed cell death by various techniques known in the art including histological and viability staining and DNA fragmentation analysis. For example, plant cells can be stained with Evans blue, a dye that is excluded from living cells with intact plasma membranes, but included in the cytoplasm of nonviable cells. Young et al., Plant Physiol. 115: 737-751; Young et al., Plant Mol. Biol. 42:397-414, Young et al. Plant Mol. Biol., 39:915-926. Levels of chlorophyll and protein in plant leaves can also be assessed to determine if plants are undergoing programmed cell death (See Lowry et al., J. Biol. Chem. 193:265-275, 1951; Peterson, Anal. Biochem. 83: 346-356, 1977; Larson et al. Anal. Biochem. 155:243-248, 1986; and U.S. Pat. No. 5,689,042). Leaves at the beginning stages of senescence show loss of chlorophyll at the tip of the leaf. Additional loss of chlorophyll and protein occurs as the leaf progresses through senescence and programmed cell death.

After cells undergoing programmed cell death are identified, RNA is extracted from the cells using methods described in the art, e.g., Puissant et al., BioTechniques 8:148-149, 1990. Poly (A+) RNA is then isolated from the extracted RNA for construction of cDNA libraries. Methods for isolating poly(A+) RNA are described in Crowell, et al, Proc. Natl. Acad. Sci. USA 87:8815-8819, 1990.

To identify genes (mRNAs) that increase in response to programmed cell death, differential screening cDNA libraries are constructed from mRNA obtained from cells undergoing programmed cell death for mRNAs that increase in abundance during programmed cell death. cDNA probes used can be made by reverse transcribing poly (A)+RNA isolated from healthy plant parts and poly (a)+RNA isolated from the same plant parts undergoing programmed cell death. For example, SAG12 and SAG13 senescence associated genes were isolated from tobacco leaves using the above-mentioned techniques, U.S. Pat. No. 5,689,042.

After programmed cell death associated mRNAs and cDNAs are isolated, promoter fragments are isolated. Typically, the promoter sequences are those from genes that are expressed in the beginning stages of programmed cell death. A number of methods are known to those of skill in the art for identifying and characterizing promoter regions in plant genomic DNA (see, e.g., Jordano, et al., Plant Cell, 1:855-866 (1989); Bustos, et al., Plant Cell, 1:839-854 (1989); Green, et al., EMBO J. 7, 4035-4044 (1988); Meier, et al., Plant Cell, 3, 309-316 (1991); and Zhang (1996) Plant Physiology 110:1069-1079). For example, programmed cell death associated promoters can be identified by analyzing the 5′ sequences of a genomic clone corresponding to a programmed cell death associated cDNA. Sequences characteristic of promoter sequences can be used to identify the promoter. Sequences controlling eukaryotic gene expression have been extensively studied. One promoter sequence element may be the TATA box consensus sequence (TATAAT), which is usually 20 to 30 base pairs upstream of the transcription start site. In most instances the TATA box is required for accurate transcription initiation. In plants, further upstream from the TATA box, at positions −80 to −100, there is typically a promoter element with a series of adenines surrounding the trinucleotide G (or T) N G. J. Messing et al., Genetic Engineering in Plants, p.221-227 (Kosage, Meredith and Hollaender, eds. (1983)). Alternatively the promoters and promoter control elements of the invention can be identified by “walking” upstream from the 5′-most portions of cDNA sequences in the genomic DNA library. Other methods, including primer extension assays, (King et al., Gene 242:125 (2000)) can be used to identify promoter regions.

Once a candidate promoter for a programmed cell death associated gene is identified, standard methods, e.g., in situ RNA hybridizations or reporter assays, can be used to determine if the putative promoter is a programmed cell death associated gene promoter. For example, in a typical reporter assay, the promoter gene is fused to a reporter gene, e.g., the beta-glucuronidase (GUS) reporter gene, and introduced into a plant of interest, e.g., maize or other grasses. The resulting plants are then fixed and assayed for expression of the reporter gene. If the reporter gene is expressed in plant parts undergoing programmed cell death but not in the same parts prior to programmed cell death, the candidate promoter can be used in the present invention.

Once a programmed cell death inducible promoter is identified from one species, e.g., SAG 12 from Arabidopsis (SEQ ID NO:1), one of skill in the art can use standard methods to identify other appropriate promoters. For example, one of skill can test promoters of other Arabidopsis SAG genes, Weaver et al., Plant Mol. Biol. 37:455-469 (1998), Noh et al., Plant Mol. Biol. 41:181-194 (1999). Alternatively, orthologs of the SAG12 or other Arabidopsis SAG genes can be identified from other species by searching EST databases. For example, after searching the maize EST database, two ESTs with significant similarity to the SAG12 gene were identified. One maize EST was obtained from developing ears (Accession Number: A1770559) whereas another was obtained from developing anthers/pollen (Accession Number: AW056019). Using the ESTs as probes, the genomic clones and promoter regions can be isolated.

Promoters useful in this invention also include promoters from genes whose expression is induced in the lower floret of the maize plant, whether associated with programmed cell death or not. For example, the Tasselseed2 promoter (Accession Number: L20621) can be used as a programmed cell death inducible promoter of this invention. Expression from the Tasselseed2 gene is induced in the lower floret of the ear and is required for lower floret cell death, DeLong et al., Cell, 74:757-768 (1993), Calderon et al., Development 126:435-441. (1999). Other genes that exhibit floret-specific expression and whose promoters can be used in this invention include the maize ZMM2 (Accession Number: X81200), ZMM6 (Accession Number: AF292703), ZMM8 (Accession Number: YO9303) and ZMM14 (Accession Number: AJ005338) genes, Cacharron et al., Dev. Genes Evol., 209:411-420. ZMM2 and ZMM6 are expressed in both the upper and lower florets of the maize plant. ZMM8 and ZMM14 are expressed in the upper floret of the maize plant. Other floret specific genes whose promoters can be used in this invention include ZAG1 (Accession Number: L18924) and ZAG2 (Accession Number: X80206) genes, Mena et al., Science 274:1537-1540. One of skill in the art would know how to identify orthologs of the floret specific genes from other plant species and isolate their promoters for use in the present invention.

IV. Construction of Expression Cassettes

The expression cassettes of this invention are used to inhibit programmed cell death in maize and other grasses. For example, the expression cassettes can be used to inhibit the programmed cell death of the lower floret in a maize spikelet. Standard methodologies can be used to prepare expression cassettes that inhibit programmed cell death by operably linking genes responsible for inhibiting programmed cell death with a programmed cell death inducible promoter. The expression cassettes can be introduced into the plants cells by a standard techniques including transformation. Techniques for transforming a wide variety of higher plant species are well known and described in the technical and scientific literature. See, for example, Gordonkamm et al, Plant Cell, V2(N7): 603-618 (1990), Ishida et al., Nature Biotechnology, V14(N6):745-750 (1996).

1. Plant growth regulators that promote programmed cell death

The skilled practitioner will know how to inhibit programmed cell death in a plant by using standard techniques to inhibit the activity of plant growth regulators that promote programmed cell death, e.g., ethylene and gibberellic acid. Methods include disrupting or knocking out genes encoding enzymes that synthesize compounds responsible for promoting programmed cell death in plants, e.g., using transposable elements to disrupt genes. Other methods include inactivating receptors that bind to the compounds responsible for promoting programmed cell death. Even other methods include degrading or conjugating the compounds or precursors to the compounds responsible for promoting programmed cell death.

One standard technique, gene silencing, can be accomplished by the introduction of a transgene corresponding to the gene of interest in the antisense orientation relative to its promoter (see, e.g., Sheehy et al., Proc. Nat'l Acad. Sci. USA 85:8805-8808 (1988); Smith et al., Nature 334:724-726 (1988)), or in the sense orientation relative to its promoter (Napoli et al., Plant Cell 2:279-289 (1990); van der Krol et al., Plant Cell 2:291-299 (1990); U.S. Pat. No. 5,034,323; U.S. Pat. No. 5,231,020; and U.S. Pat. No. 5,283,184), both of which lead to reduced expression of the transgene as well as the endogenous gene.

Posttranscriptional gene silencing has been reported to be accompanied by the accumulation of small (20-25 nucleotide) fragments of antisense RNA, which are reported to be synthesized from an RNA template and represent the specificity and mobility determinants of the process (Hamilton & Baulcombe, Science 286:950-952 (1999)). It has become clear that in a range of organisms the introduction of dsRNA (double-stranded RNA) is an important component leading to gene silencing (Fire et al., Nature 391:806-811 (1998); Timmons & Fire, Nature 395:854 (1998); WO99/32619; Kennerdell & Carthew, Cell 95:1017-1026 (1998); Ngo et al., Proc. Nat'l Acad. Sci. USA 95:14687-14692 (1998); Waterhouse et al, Proc. Nat'l Acad. Sci. USA 95:13959-13964 (1998); WO99/53050; Cogoni & Macino, Nature 399:166-169 (1999); Lohmann et al., Dev. Biol. 214:211-214 (1999); Sanchez-Alvarado & Newmark, Proc. Nat'l Acad. Sci. USA 96:5049-5054 (1999)). In plants, the suppressed gene does not need to be an endogenous plant gene, since both reporter transgenes and virus genes are subject to posttranscriptional gene silencing by introduced transgenes (English et al., Plant Cell 8:179-188 (1996); Waterhouse et al., supra). However, in all of the above cases, some sequence similarity is required between the introduced transgene and the gene that is suppressed.

High frequency and high level posttranscriptional gene silencing have been found by introduction either of constructs containing inverted repeats of the coding regions of virus or reporter genes, or by crossing together plants expressing the sense and antisense transcripts of the coding region of the target gene (Waterhouse et al., Proc. Nat'l Acad. Sci. USA 95:13959-13964 (1998)). Similar results are obtained by expression of sense and antisense transgenes under the control of different promoters in the same plant (Chuang & Meyerowitz, Proc. Nat'l Acad. Sci USA 97:4985-4990 (2000)).

In one example, a nucleic acid segment from a gene that synthesizes compounds responsible for promoting programmed cell death is cloned and operably linked to a programmed cell death associated promoter such that the antisense strand of RNA will be transcribed. The expression cassette is then transformed into maize or other grasses and the antisense RNA strand is produced. The antisense RNA inhibits gene expression in the cells by preventing the accumulation of mRNA which encodes the enzyme of interest, see, e.g., Sheehy et al., Proc. Nat. Acad. Sci USA, 85:8805-8809 (1988), and Hiatt et al., U.S. Pat. No. 4,801,340.

The nucleic acid segment to be introduced is substantially identical to at least a portion of the endogenous gene or genes to be repressed. The sequence, however, need not be perfectly identical to inhibit expression. The expression cassettes of the present invention can be designed such that the inhibitory effect applies to others proteins within a family of genes exhibiting homology or substantial homology to the target gene.

The introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides should be used, though a sequence of at least about 100, 200 or 500 nucleotides is preferred.

In another example, a nucleic acid segment from a gene that synthesizes compounds or plant growth regulators responsible for promoting programmed cell death is cloned and operably linked to a programmed cell death associated promoter such that the sense strand of RNA will be transcribed.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% or about 95% identity is preferred. As with antisense regulation, the effect should apply to any other proteins within a similar family of genes exhibiting homology or substantial homology.

For sense suppression, the introduced sequence in the expression cassette needing less than absolute identity, also need not be full length, relative to either the primary transcription product or fully processed mRNA. This may be preferred to avoid concurrent production of some plants which are overexpressers. A higher identity in a shorter than full length sequence compensates for a longer, less identical sequence. Furthermore, the introduced sequence need not have the same intron or exon pattern, and identity of non-coding segments will be equally effective. Normally, a sequence of the size ranges notes above for antisense regulation is used.

Catalytic RNA molecules or ribozymes can also be used to inhibit expression of genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs which are capable of self-cleavage and replication in plants. The RNAs replicate ether alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described in Haseloff et al., Nature, 334: 585-591 (1988).

2. Plant growth regulators that inhibit programmed cell death

As with plant growth regulators that promote programmed cell death, the present invention also provides methods of inhibiting programmed cell death in a plant by modulating the activity of compounds, e.g., cytokinin and abscisic acid, that inhibit programmed cell death.

Standard techniques can be used to influence the activity of compounds responsible for inhibiting programmed cell death. These techniques include increasing expression of enzymes that synthesize compounds responsible for inhibiting programmed cell death, e.g., a gene that encodes isopentenyl transferase can be linked to a programmed cell death inducible promoter and introduced into a maize plant. Isopentenyl transferase catalyzes the synthesis of cytokinin, a hormone that inhibits programmed cell death in the lower floret of a maize spikelet. Examples of IPT sequences are presented in: Crespi et al., EMBO J. 11:795-804 (1992); Goldberg et al., Nucleic Acids. Res. 12:4665-4677 (1984); Heide Kamp et al., Nucleic Acids Res., 11:6211-6223 (1983); Strabala et al., Mol. Gen. Genet. 216:388-394 (1989). Accession Number: NC_{—}003308. Other methods of influencing compounds responsible for inhibiting programmed cell death are known in the art and include inhibiting expression of enzymes that metabolize compounds that inhibit programmed cell death.

V. Detection of Kernels with Multiple Embryos

After preparation of the expression cassettes of the present invention and introduction of the cassettes into maize, one of skill in the art would know how to detect the presence of a kernel with multiple embryos and increased protein and oil content. For example, after introduction of the cassette into maize, the plants are screened for the presence of the transgene and crossed to a maize inbred or hybrid line. Progeny plants are then screened for presence of the transgene and self-pollinated. Progeny from the self-pollinated plants are grown. The kernels of the progeny are examined and those that contain the transgene contain kernels with multiple embryos.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLE 1

In order to introduce an isopentenyl transferase (IPT) gene into maize whose expression would be specifically induced in the lower floret prior to or concomitant with the onset of its programmed cell death, pSG516 (Gan et al., Science, 270(5244) p.1986-8), a construct in which the IPT gene is under the control of the promoter from the Arabidopsis senescence-associated gene (SAG 12) gene, was used.

The SAG12-IPT construct (pSG516) was introduced into embryogenic callus obtained from developing embryos of the maize line, HiII, using particle bombardment. The Streptomyces hygroscopicus bar containing plasmid construct was co-bombarded with pSG516. Expression from the bar gene produces phosphinothricin acetyltransferase (PAT) which inactivates the herbicide phosphinothricin (PPT). Thus cells or plants expressing bar are resistant against glufosinate (the ammonium salt of PPT) or bialaphos (which contains PPT). Bialophos-resistant calli were grown and plants were regenerated according to standard procedures (Gordon-Kamm et al., 1990).

Regenerated plants were allowed to flower and crossed to the inbred B73. Progeny from this cross were hemizygous for the SAG12-IPT construct and once grown, were self-pollinated. Kernels from this pollination exhibited two embryos with a fused endosperm and segregated with the segregating population. Progeny from this pollination containing the SAG12-IPT transgene were grown and self-pollinated. Rescue of up to 40% of the lower florets of developing ears was observed.

The above example is provided to illustrate the invention but not to limit its scope. Other variants of this invention will be readily apparent to one of ordinary skill in the art and are encompassed by the appended claims.

What is claimed is:

1. A method of inhibiting programmed cell death in a maize plant comprising introducing a construct comprising a programmed cell death inducible promoter operably linked to a nucleotide sequence that inhibits programmed cell death into said plant, whereby programmed cell death in the lower floret of said plant is inhibited.

2. The method of claim 1, wherein the nucleotide sequence encodes a plant growth regulator synthesizing enzyme.

3. The method of claim 2, wherein the enzyme catalyzes the synthesis of cytokinin.

4. The method of claim 3, wherein the enzyme is isopentenyl transferase.

5. The method of claim 1, wherein the programmed cell death inducible promoter is SAG12.

6. The method of claim 5, wherein the SAG12 promoter is from Arabidopsis thaliana.

7. The method of claim 6, wherein the SAG12 promoter is 70% identical to SEQ ID NO:1.

8. The method of claim 1, further comprising detecting increased levels of protein within said plant.

9. The method of claim 1, further comprising detecting increased levels of oil within said plant.

10. The method of claim 1, further comprising detecting increased levels of oil and protein within said plant.

11. The method of claim 1, further comprising detecting the presence of a kernel having multiple embryos.

12. The method of claim 1, wherein the construct is introduced by a type of sexual cross.

13. The method of claim 1, wherein the construct is introduced by transformation.

14. A transgenic maize plant comprising an expression cassette comprising a programmed cell death-inducible promoter operably linked to a nucleotide sequence encoding an inhibitor of programmed cell death, the maize plant having kernels with multiple embryos.

15. The transgenic plant of claim 14, wherein the nucleotide sequence encodes a plant growth regulator synthesizing enzyme.

16. The transgenic plant of claim 15, wherein the enzyme catalyzes the synthesis of cytokinin.

17. The transgenic plant of claim 16, wherein the enzyme is isopentenyl transferase.

18. The transgenic plant of claim 14, wherein the programmed cell death inducible promoter is SAG12.

19. A kernel from a transgenic maize plant comprising multiple embryos, wherein the kernel has increased oil and protein content.

20. A method of inhibiting programmed cell death in a maize plant comprising introducing a promoter from a floret specific gene operably linked to a nucleotide sequence that inhibits programmed cell death into said plant, whereby programmed cell death in the lower floret of said plant is inhibited.

21. The method of claim 20, wherein the floret specific gene is associated with programmed cell death.

22. The method of claim 20, wherein the floret specific gene is not associated with programmed cell death

23. The method of claim 20, wherein the nucleotide sequence encodes a plant growth regulator synthesizing enzyme.

24. The method of claim 23, wherein the enzyme catalyzes the synthesis of cytokinin.

25. The method of claim 24, wherein the enzyme is isopentenyl transferase.

26. The method of claim 20, further comprising detecting increased levels of oil and protein within said plant.

27. The method of claim 20, further comprising detecting the presence of a kernel having multiple embryos.


SEQUENCE LISTING (1 sequence)

SEQ ID NO: 1 - DNA, 3183 bp, Arabidopsis thaliana
  promoter (1)..(3183): senescence-associated gene 12-1 (SAG12-1) promoter
gatatctctttttatattcaaacaataagttgagatatgtttgagaagaggacaactattctcgtggagcaccgagtctgttttatattagaaacccgattgttatttttagactgagacaaaaaagtaaaatcgttgattgttaaaatttaaaattagtttcatcacgtttcgataaaaaaatgattagttatcatagctaatatagcatgattctaaatttgttttttgacaccctttttttctctctttggtgttttcttaacattagaagaacccataacaatgtacgttcaaattaattaaaaacaatatttccaagttttatatacgaaacttgtttttttaatgaaaacagttgaatagttgattatgaattagttagatcaatactcaatatatgatcaatgatgtatatatatgaactcagttgttatacaagaaatgaaaatgctatttaaataccgatcatgaagtgttaaaaagtgtcagaatatgacatgaagcgttttgtcctaccgggtatcgagttataggtttggatctctcaagaatattttgggccatattagttatatttgggcttaagcgttttgcaaagagacgaggaagaaagattgggtcaagttaacaaaacagagacactcgtattagttggtactttggtagcaagtcgatttatttgccagtaaaaacttggtacacaactgacaactcgtatcgttattagtttgtacttggtacctttggttaagaaaaagttgatatagttaaatcagttgtgttcatgaggtgattgtgatttaatttgttgactagggcgattccttcacatcacaataacaaagttttatagatttttttttataacatttttgccacgcttcgtaaagtttggtatttacaccgcatttttccctgtacaagaattcatatattatttatttatatactccagttgacaattataagtttataacgtttttacaattatttaaataccatgtgaagatccaagaatatgtcttacttcttctttgtgtaagaaaactaactatatcactataataaaataattctaatcattatatttgtaaatatgcagttatttgtcaattttgaatttagtattttagacggttatcacttcagccaaatatgatttggatttaagtccaaaatgcaatttcgtacgtatccctcttgtcgtctaatgattatttcaatatttcttatattatccctaactacagagctacatttatattgtattctaatgacagggaaactttcatagagattcagatagatgaaattggtgggaaacatcattgaacaggaaacttttagcaaatcatatcgatttatctacaaaagaatacttagcgtaatgaagttcacttgttgtgaatgactatgatttgatcaaattagttaattttgtcgaatcatttttctttttgatttgattaagcttttaacttgcacgaatggttctcttgtgaataaacagaatctttgaattcaaactatttgattagtgaaaagacaaaagaagattccttgtttttatgtgattagtgattttgatgcatgaaaggtacctacgtactacaagaaaaataaacatgtacgtaactacgtatcagcatgtaaaagtatttttttccaaataatttatactcatgatagattttttttttttgaaatgtcaattaaaaatgctttcttaaatattaattttaattaattaaataaggaaatatatttatgcaaaacatcatcaacacatatccaacttcgaaaatctctatagtacacaagtagagaaaataaattttactagatacaaacttcctaatcatcaattataaatgtttacaaaactaattaaacccaccactaaaattaactaaaaatccgagcaaagtgagtgaacaagacttgatttcaggttgatgtaggactaaaatggctacgtatcaaacatcaacgatcatttagttatgtatgaatgaatgtagtcattacttgtaaaacaaaaatgctttgatttggatcaatcacttcatgtgaacattagcaattacatcaaccttattttcactataaaaccccatctcagtacccttctgaagtaatcaaattaagagcaaaagtcatttaactttcctaaaaccatggaccctgcatctaattttcggtccaacttgcacaggaaagacgacgaccgcgatagctcttgcccagcagacagggcttccagtcctttcgcttgatcgggtccaatcgtgtcctcaactatcaaccggaagcggacgaccaacagtggaagaactgaaaggaacgacgcgtctctaccttgatgatcggcctctggtggagggtatcatcgcagccaagcaagctcatcataggctgatcgaggaggtgtataatcatgaggccaacggcgggcttattcttgagggaggatccacctcgttgctcaactgcatggcgcgaaacagctattggagtgcagattttcgttggcatattattcgccacaagttacccgaccaagagaccttcatgaaagcggccaaggccagagttaagcagatgttgcaccccgctgcaggccattctattattcaagagttggtttatctttggaatgaacctcggctgaggcccattctgaaagagatcgatggatatcgatatgccatgttgtttgctagccagaaccagatcacggcagatatgctattgcagcttgacgcaaatatggaaggtaagttgattaatgggatcgctcaggagtatttcatccatgcgcgccaacaggaacagaaattcccccaagttaacgcagccgctttcgacggattcgaaggtcatccgttcggaatgtattaggttacgccagccctgagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatggcgcgcaaactgggataaattatcgcgcgcggtgtcatctatgttactagatcgaattc